(19) United States
(12) Patent Application Publication
(10) Pub. No.: US 2026/0158240 A1
(43) Pub. Date: Jun. 11, 2026

(54) INTRAVENOUS MONITORING SYSTEM

(71) Applicant: CareFusion 303, Inc., San Diego, CA (US)

(72) Inventors: Gene E. MASON, La Habra Heights, CA (US); Likhith NALLURI, Brea, CA (US); Sara RUTH, Englewood, NJ (US); Shishir PRASAD, Ramsey, NJ (US)

(21) Appl. No.: 18/970,157

(22) Filed: Dec. 5, 2024

(51) Int. Cl. A61M 25/06; A61M 25/00

(52) U.S. Cl. CPC A61M 25/0606; A61M 25/0097; A61M 2205/18; A61M 2205/3303; A61M 2205/3327; A61M 2205/3331; A61M 2205/3561; A61M 2205/3592; A61M 2205/50; A61M 2205/581; A61M 2205/583; A61M 2205/8206; A61M 2230/30

(57) ABSTRACT

Monitoring systems are disclosed that can include a fluid pathway formed in a housing to receive and direct a fluid through the housing, a sensor coupled to the fluid pathway to detect data for a fluid in the fluid pathway, a communication unit for conveying data sensed by the sensor, and a power source, where the systems of the present disclosure can be used for intravenous monitoring when coupled to a catheter or intravenous set, or can include a catheter needle extending from the housing, and in which the systems of the systems of the present disclosure can detect characteristics such as placement of a catheter, fluid pathway occlusion or patency, and catheter extravasation or infiltration.

### BACKGROUND

The present disclosure generally relates to monitoring characteristics associated with intravenous fluid flow, and in particular, to intravenous monitoring systems having a sensor and communication unit.

Infusion therapy is a common healthcare procedure that may be facilitated by a vascular access device. Patients in hospital, home care, and other setting may receive fluids, medication, and blood products through a vascular access device. Additionally, blood may be withdrawn from the patient using the vascular access device. Blood withdrawal is another common healthcare procedure that may be facilitated by the vascular access device.

The vascular access device may be inserted into a peripheral or central vasculature of a patient. The vascular access device may be indwelling for short term (e.g., days), moderate term (e.g., weeks), or long term (e.g., months to years). The vascular access device may be used for continuous infusion therapy or for intermittent infusion therapy. A common type of vascular access device is a catheter, such as, for example, a peripheral intravenous catheter (PIVC) or a peripherally inserted central catheter (PICC).

A common type of catheter is an over-the-needle PIVC. The over-the-needle PIVC may be mounted over an introducer needle having a sharp distal tip. The PIVC and the introducer needle may be assembled so that the distal tip of the introducer needle extends beyond the distal tip of the PIVC with the bevel of the needle facing away from skin of the patient. The PIVC and the introducer needle are generally inserted at a shallow angle through the skin and into a blood vessel of the patient, such as an artery, a vein, or any other vasculature of the patient.

To verify proper placement of the PIVC within a blood vessel, a clinician will typically watch for blood “flashback” to occur within the PIVC. Blood flashback occurs when blood travels proximally between an outer surface of the introducer needle and an inner surface of the PIVC, and to a flashback window or flashback chamber. Thus, the clinician may visualize the blood and confirm placement of the introducer needle within the vasculature. Once placement of the needle has been confirmed, the clinician may temporarily occlude flow in the vein and remove the introducer needle, leaving the PIVC in place for future blood withdrawal and/or fluid infusion.

Once a PIVC has been placed within a blood vessel and a drug is delivered to the blood vessel via the PIVC, extravasation of the drug out of the blood vessel and into soft tissues adjacent the blood vessel may occur. A sensor may be attached to the patient's skin close to the catheter insertion site to monitor the patient for drug extravasation events. However, this method of detection may only work after a significant amount of the drug has leaked out of the catheter/blood vessel and migrates close to the patient's skin. Moreover, a sensor placed on the patient's skin is an additional attachment to the patient and cost which has to be removed and re-attached periodically, such as when the patient goes to the bathroom or bathes.

Procedures for verifying proper placement of the PIVC, fluid pathway occlusion or patency, and catheter extravasation or infiltration typically require visual observation, consideration, and judgement by a clinician. In some instances, the clinician may determine the degree of occlusion in the catheter by manually applying pressure to the catheter from a syringe or observing the degree of difficulty to withdraw blood from the catheter. Upon determining that an issue or problem exists with a catheter or other device coupled to the catheter, such as fluid pathway occlusion, extravasation, or infiltration, the catheter may be removed, replaced, or an enzyme may be introduced to restore fluid flow through catheter. In some instances, an issue or problem associated with the catheter may be identified inaccurately or an untimely manner using conventional procedures such as visual observation or manual examination of the IV tubing and catheter.

### SUMMARY

The disclosed subject matter relates to intravenous monitoring systems that can be coupled with an IV set or catheter to detect characteristics associated with any of the IV set or catheter, including placement of a PIVC, fluid pathway occlusion or patency, extravasation, and infiltration. The intravenous monitoring systems of the present disclosure provide for evaluation of IV set or catheter to determine if a problem or failure may occur or has occurred. In this manner, the intravenous monitoring systems permit timely identification of symptoms and potential or existing issues. Timely identification of symptoms and issues can permit a clinician, caregiver, or other actor to correct the condition causing the issue, reposition the IV set or catheter, or otherwise replace the IV set or catheter.

In some embodiments, an intravenous monitoring system is disclosed that comprises a housing having an inner surface forming a fluid pathway extending through the housing and configured for coupling with a catheter, a sensor coupled to the fluid pathway to detect data for a fluid in the fluid pathway, a communication unit coupled to the sensor for conveying data sensed by the sensor, and a power source electrically coupled to the sensor.

In certain embodiments, an intravenous monitoring system is disclosed and comprises a housing having a first end, a second end opposite to the first end, a longitudinal axis formed between the first end and the second end, and an inner surface forming a fluid pathway extending through the housing and configured for coupling with a catheter, a sensor coupled to the fluid pathway to detect data for a fluid in the fluid pathway, a communication unit coupled to the sensor for conveying data sensed by the sensor, and a power source electrically coupled to the sensor.

In some instances, the present disclosure provides an intravenous monitoring system comprising a catheter having catheter hub and a catheter needle, the catheter hub comprising a housing having an inner surface forming a fluid pathway extending through the housing, and the catheter needle having a proximal end coupled to the housing, a distal end, and an inner surface forming a lumen extending through the catheter needle and fluidically coupled to the fluid pathway, a sensor coupled to the housing to detect data for a fluid in any of the fluid pathway and the lumen, a communication unit coupled to the housing for conveying data sensed by the sensor, and a power source coupled to the housing and electrically coupled to the sensor.

In some instances, methods for providing intravenous monitoring systems are disclosed as providing a housing comprising a first end, a second end opposite to the first end, and an inner surface forming a fluid pathway extending through the housing and configured for fluidically coupling with a catheter, providing a sensor coupled to the fluid pathway to detect data for a fluid in the fluid pathway, providing a communication unit coupled to the sensor for conveying data sensed by the sensor, and providing a power source electrically coupled to the sensor.

Accordingly, the present application addresses several operational challenges for evaluating an IV set, IV tubing, or a catheter, including proper placement, fluid pathway occlusion or patency, and extravasation or infiltration, and provides numerous improvements that enable increased accuracy, timeliness, safety, and efficacy for clinicians and patients.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates an embodiment of an intravenous monitoring system in use with a PIVC catheter coupled to a patient, in accordance with aspects of the present disclosure.

FIG. 2 illustrates another embodiment of an intravenous monitoring system in use with a PIVC catheter coupled to a patient, in accordance with aspects of the present disclosure.

FIG. 3 illustrates another embodiment of an intravenous monitoring system in use with a PIVC catheter coupled to a patient, in accordance with aspects of the present disclosure.

FIG. 4 is a top perspective view of the intravenous monitoring system of FIG. 1, in accordance with aspects of the present disclosure.

FIG. 5 is a side cross-sectional view of the intravenous monitoring system of FIG. 4 along the line 5-5, in accordance with various aspects of the present disclosure.

FIG. 6 is a top perspective view of the intravenous monitoring system of FIG. 2, in accordance with aspects of the present disclosure.

FIG. 7 is a side cross-sectional view of the intravenous monitoring system of FIG. 6 along the line 7-7, in accordance with various aspects of the present disclosure.

FIG. 8 is illustrates an intravenous monitoring system in use with a PIVC catheter coupled to a patient, in accordance with aspects of the present disclosure.

FIG. 9 is a top perspective view of the intravenous monitoring system of FIG. 3, in accordance with aspects of the present disclosure.

### DETAILED DESCRIPTION

The disclosed intravenous monitoring systems incorporate various features to facilitate the detection of characteristics and evaluation of several aspects of venous access, including proper position of a catheter inside a vein, fluid pathway occlusion or patency, and catheter extravasation or infiltration.

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology. Further, while the present description sets forth specific details of various embodiments, it will be appreciated that the description is illustrative only and should not be construed in any way as limiting.

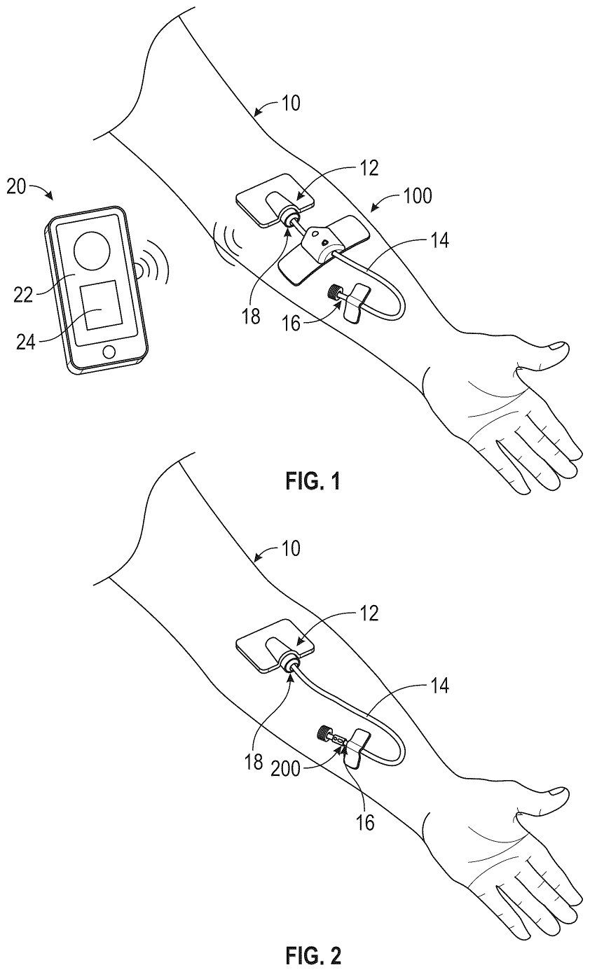

Referring now to the drawings, FIG. 1 illustrates an example of an intravenous monitoring system 100 coupled with a patient 10, according to some embodiments. The intravenous monitoring system 100 is coupled to a catheter 12 and tubing 14, where the tubing is coupled to the intravenous monitoring system 100 such that a fluid (e.g., blood) moving through the tubing 14 will move through a fluid pathway of the intravenous monitoring system 100.

The intravenous monitoring system 100 can be coupled to the tubing 14 by inserting the tubing through the intravenous monitoring system 100 such that the intravenous monitoring system 100 is positioned between a first end 16 of the tubing and a second end 18 of the tubing. In some embodiments, the intravenous monitoring system 100 can be coupled between separate segments of tubing. For example, a first end of a first tubing segment can be coupled to the intravenous monitoring system 100 and a second end of the first tubing segment can be coupled to the catheter 12, and a first end of a second tubing segment can be coupled to another portion of the intravenous monitoring system 100 and a second end of the second tubing segment can be coupled to another device such as a Luer lock adapter. When coupled to the intravenous monitoring system 100, the first end of a first tubing segment and the first end of a second tubing segment are in fluid communication with the fluid pathway of the intravenous monitoring system 100.

The intravenous monitoring system 100 can be removably coupled to the tubing 14 when the catheter is prepared for insertion into the patient, thereby minimizing any disruption to current workflow practice of a caregiver. The present disclosure also contemplates that the intravenous monitoring system 100 can be coupled to any of the catheter 12 and the tubing 14 in a manner that resists removal of the intravenous monitoring system 100 via any suitable coupling mechanism, including, for example, threading, a snap fit, an interference fit, friction, or an adhesive.

The intravenous monitoring system 100 can be configured as an anchor or catheter securement device. To facilitate anchoring or securing the catheter to the patient, and to resist unintended separation of the catheter from the patient, the intravenous monitoring system 100 can be configured to be coupled to the patient by taping the intravenous monitoring system 100 to the patient or applying an adhesive between the intravenous monitoring system 100 and the patient. If a portion of the tubing 14, such as the first end 16 of the tubing, is pulled in a direction away from the catheter 12, an intravenous monitoring system 100 configured as a catheter securement device that is secured to the patient can resist separation of the catheter from the patient by resisting transfer of a force to the catheter as a result of the tubing being pulled.

FIG. 2 illustrates an exemplary embodiment of an intravenous monitoring system 200 fluidically coupled to a catheter 12 that is inserted into a patient 10. The intravenous monitoring system 200 includes features that are similar to the intravenous monitoring system 100 described and/or shown in other portions of the present disclosure. Accordingly, certain features of the intravenous monitoring system 200 are referred to with similar reference numerals used in reference to the intravenous monitoring system 100.

The intravenous monitoring system 200 is fluidically coupled to a catheter 12 such that a fluid (e.g., blood) moving through the tubing 14 will move through a fluid pathway of the intravenous monitoring system 200. To fluidically couple the intravenous monitoring system 200 to the catheter 12, a first end 16 of the tubing is coupled to a first portion of the intravenous monitoring system 200 and is in fluid communication with the fluid pathway of the intravenous monitoring system, and a second end 18 of the tubing is coupled to the catheter 12. A second portion of the intravenous monitoring system 200 can be configured to couple to another segment of tubing or another device such as a needleless connector, a Luer lock adapter, a syringe, or a blood collection device.

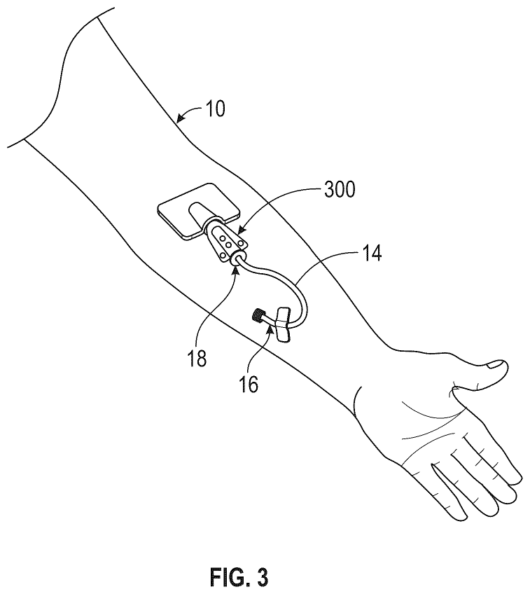

FIG. 3 illustrates an exemplary embodiment of an intravenous monitoring system 300 configured as a catheter hub with a catheter needle that is inserted into a patient 10. The intravenous monitoring system 300 includes features that are similar to the intravenous monitoring systems 100, 200 described and/or shown in other portions of the present disclosure. Accordingly, certain features of the intravenous monitoring system 300 are referred to with similar reference numerals used in reference to the intravenous monitoring systems 100, 200.

The intravenous monitoring system 300 includes a first or proximal end and a second or distal end. The first end of the intravenous monitoring system 300 is coupled to a second end 18 of the tubing, and the second end of the intravenous monitoring system is coupled to or comprises a catheter needle. Each of the tubing 14 and the catheter needle are in fluid communication with the fluid pathway of the intravenous monitoring system.

In any of the embodiments of the present disclosure, including the intravenous monitoring systems 100, 200, 300, the intravenous monitoring system can include a housing that can be coupled with any of an IV tubing and/or a catheter. The housing includes an inner surface that forms a fluid pathway extending through the housing to permit a fluid to move through the housing. To sense, collect, and identify data associated with the fluid moving through the housing, the intravenous monitoring system includes one or more sensor and a communication unit. The sensor is coupled to the fluid pathway and can be in indirect contact or direct contact with the fluid in the fluid pathway. The sensor can be coupled to the housing or the sensor can be positioned within a cavity formed the housing. Additionally, the intravenous monitoring system can include more than one sensor and more than one communication unit.

The sensor can detect conditions and characteristics of a fluid in the fluid pathway, such as a change in pressure caused by the fluid. In instances where the intravenous monitoring system is in fluid communication with a fluid pathway extending through a catheter, the sensor can detect intravascular pressure changes such as peripheral venous pressure (PVP) waveform. Thus, by detecting conditions within the fluid pathway, the sensor may detect conditions within the fluid pathway extending through the catheter 12. In some embodiments of the present disclosure, a sensor of the intravenous monitoring system can detect the presence or absence of PVP waveform.

The one or more sensor of the intravenous monitoring system may include any of a pressure sensor, a temperature sensor, and/or a flow sensor. In some embodiments, a pressure sensor may be configured to detect a fluid pressure of fluid within the fluid pathway. In some embodiments, a flow sensor may be configured to detect a fluid flow rate and/or a fluid flow volume within the fluid pathway.

The pressure sensor may include any suitable pressure sensor that can detect fluid pressure within the fluid pathway. In some embodiments, the pressure sensor can include a pressure sensitive device, which may be any of capacitive, resistive, optical, and/or ultrasonic. In some embodiments, a first surface of the pressure sensor may be exposed to the fluid within the fluid pathway and a second surface of the pressure sensor may be exposed to a liquid or gas at a reference pressure, thereby providing a reference surface. In some embodiments, the measured pressure differential between the first surface and the second surface of the pressure sensor can provide an indication of the fluid pressure to which the first surface is exposed.

In instances in which the intravenous monitoring system includes a flow sensor, the flow sensor can include any suitable flow sensor that can detect fluid flow through the fluid pathway. Although various suitable fluid flow sensors are well known and may be used, illustrative examples of suitable fluid flow sensors can include, but are not limited to, optical sensors, piezoelectric sensors, sound sensors, reed switch-based sensors, magnetic sensors, ultrasound sensors, orifice type flow meters, venturi flow meters, and the like.

The intravenous monitoring system can also include a communication unit that generates an output signal based on the data sensed or collected by the sensor. The output signal can include one or more various formats, including, but not limited to, a visual signal, an audible signal, a tactile signal, and/or a wirelessly transmitted output signal. The visual signal, as an example, may be a light source that can be visible to a person near the intravenous monitoring system. The audible signal, as an example, may be a speaker or a piezo alarm that can be heard by a person near the intravenous monitoring system. The tactile signal, as an example, may be a vibration motor or haptic actuator that can be felt by a person in contact with the intravenous monitoring system. In some embodiments, the intravenous monitoring system can alert the patient, a caregiver, another person, or a receipt location of a catheter issue or condition by communicating any of a different light color or pattern, different sound color or pattern, or different haptic or vibration pattern, for each condition or problem. Additionally, the present disclosure contemplates that any embodiment of the intravenous monitoring system can include wirelessly transmitting the output signal from the intravenous monitoring system 100, 200, 300 to a receipt location 20.

The intravenous monitoring system of the present disclosure can also include a reset command, switch, or button that can be activated by any of patient, a caregiver, another person, or from another system associated with the intravenous monitoring system. In some embodiments of the present disclosure, a reset can be used to change a characteristic of the output signal, such as, for example, silencing, turning off, snoozing, or resetting the output signal. In some embodiments, the reset can also include other functions, such as turning the intravenous monitoring system off or on, or resetting/restarting the intravenous monitoring system.

In some embodiments, a circuit, such as a printed circuit board (“PCB”) and/or a power supply is disposed within the housing. The power supply may include a battery, which may be rechargeable and/or replaceable. In some embodiments, a location of the circuit and/or the power supply within the housing may vary. The power supply may be electrically coupled to the sensor and may be configured to power the one or more sensors. In some embodiments, the power supply may be remotely disposed from the circuit. In some embodiments, a non-volatile memory storage location, such as flash memory for instance, may be included on the circuit to enable data sensed by the sensors to be temporarily or permanently stored thereon. In some embodiments, the storage location may be accessible by a user and/or can be transmitted to the receipt location.

In some embodiments, any of the communication unit and a processor may be disposed on the circuit, which may be electrically coupled to the one or more sensor. In some embodiments, presence of an occlusion in the catheter 12 may be determined based on the data sensed by the one or more sensor. In some embodiments, the occlusion may partially block fluid flow through the catheter 12, or may fully or substantially fully block fluid flow through the catheter 12. In some embodiments, in response to the determining the presence of the occlusion based on the data sensed by the sensor, the communication unit may transmit the output signal. The output signal, may be transmitted as in one or more format, including as a wireless signal to the receipt location, a visual signal (e.g., LED light), and audio signal (e.g., alarm sound), and a tactile signal (e.g., vibration mechanism).

In some embodiments, in response to receipt of the output signal by the receipt location 20, an alert may be provided at the receipt location 20. In some embodiments, the alert from any of the intravenous monitoring system and the receipt location may include a visual signal, an audible signal, and/or a tactile signal, such as, for example, a change in status of a light or a beep sound. In some embodiments, an indicator at the receipt location may be configured to provide the alert. Alerts can also be configured for each alert type to represent a specific catheter related complication.

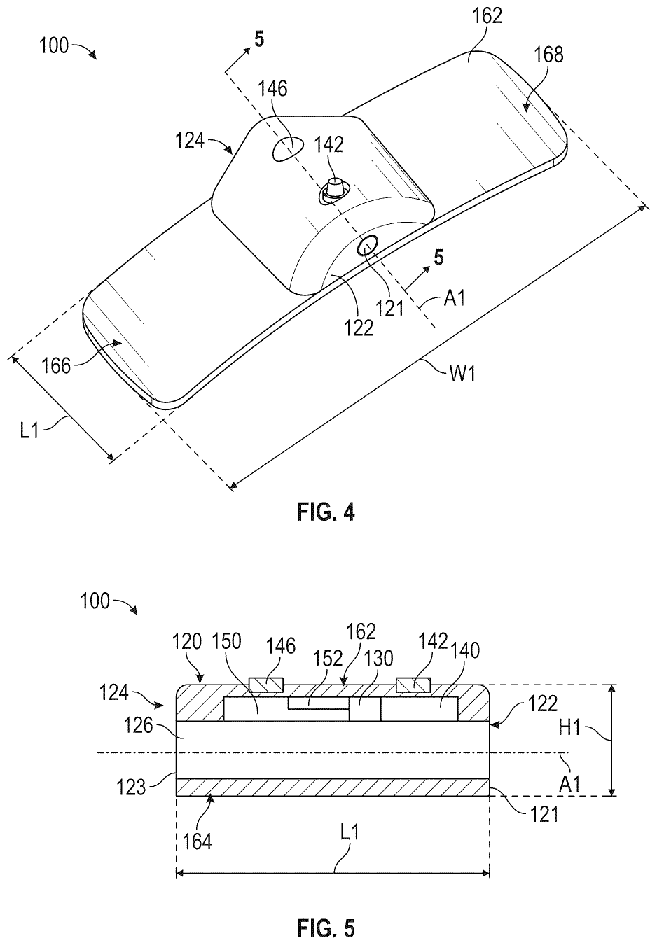

A perspective view of an embodiment of the intravenous monitoring system 100 configured as a catheter securement device is shown in FIG. 4, and a cross-section side view of the intravenous monitoring system 100 along the line 5-5 is shown in FIG. 5. The intravenous monitoring system 100 includes a housing 120 with a fluid pathway that can be coupled with IV tubing, and the intravenous monitoring system 100 can be coupled to a patient to resist separation of the catheter from the patient if the IV tubing is pulled in a direction away from the catheter. The housing 120 includes a first end 122 and a second end 124, where the second end 124 is opposite to the first end 122 of the housing. The housing 120 also includes an inner surface forming a fluid pathway 126 that extends through the housing 120 and from the first end 122 to the second end 124 of the housing. The fluid pathway 126 permits a fluid, such as blood, to move through the fluid pathway 126. When the fluid is in the fluid pathway 126, a sensor of the intravenous monitoring system 100 can sense and collect data associated with the fluid.

To sense and collect data associated with the fluid in the fluid pathway 126, the intravenous monitoring system 100 includes a sensor 130 and a communication unit 140. The sensor 130 is coupled to the fluid pathway 126 and can be in indirect or direct contact with the fluid in the fluid pathway 126. The sensor 130 can detect conditions and characteristics of the fluid in the fluid pathway 126, such as a change in pressure and/or temperature caused by the fluid. When the fluid pathway 126 is in fluid communication with a catheter, the sensor 130 can detect intravascular pressure changes such as peripheral venous pressure (PVP) waveform.

The sensor 130 of the intravenous monitoring system 100 includes a pressure sensor that can detect pressure of a fluid within the fluid pathway 126. The sensor 130 can detect conditions and characteristics of the fluid in the fluid pathway 126 by either inserting the tubing 14 through the fluid pathway 126 and through the housing 120, or by coupling an end of the tubing to the housing 120 so that the fluid can move from the tubing into and through the fluid pathway 126 of the housing 120.

The intravenous monitoring system 100 also includes a communication unit 140 that generates an output signal based on the data sensed by the sensor 130. The output signal can include one or more formats, including, but not limited to, a visual signal, an audible signal, a tactile signal, and/or a wirelessly transmitted output signal. In the embodiment of the intravenous monitoring system 100, the visual signal is a light source 142 that can be visible to a person or device that is near the intravenous monitoring system. In some embodiments, the intravenous monitoring system can alert the patient, a caregiver, another person, or a receipt location of a catheter issue or condition by communicating a different light color or light pattern, different sound or sound pattern, or different haptic or vibration pattern, for each condition or problem that is detected by the system. Additionally, the communication unit can wirelessly transmit an output signal from the intravenous monitoring system 100 to a receipt location 20 or another device.

The intravenous monitoring system 100 also includes a reset button 146 that can be activated by any of patient, a caregiver, another person, or from another system associated with the intravenous monitoring system. The reset button 146 can be used to change a characteristic of the output signal, such as, for example, silencing, turning off, snoozing, or resetting the output signal. In some embodiments, the reset button 146 can also include other functions, such as turning the intravenous monitoring system 100 off or on, or resetting/restarting the intravenous monitoring system 100.

Within the housing 120, the intravenous monitoring system 100 contains a circuit 150 and a power supply 152, which is shown, for example, in FIG. 5. In some embodiments, a non-volatile memory storage location, such as flash memory for instance, may be included on the circuit 150 to enable data sensed by the sensors to be temporarily or permanently stored thereon. In some embodiments, the storage location may be accessible by a user and/or can be transmitted to the receipt location.

The sensor 130, the communication unit 140, the light source 142, and the power supply 152 are coupled to the circuit 150. The power supply 152 is electrically coupled to the sensor 130 and provides power to the one or more sensors. The power supply 152 may include a battery, which may be rechargeable and/or replaceable. In some embodiments, the power supply 152 may be remotely disposed from the circuit 150.

Any of the communication unit 140 and a processor may be disposed on the circuit 150, which may be electrically coupled to the sensor 130. In some embodiments, the presence of an occlusion in the catheter 12 may be determined based on the data sensed by the sensor 130. The occlusion may partially block fluid flow through the catheter 12, or may fully or substantially fully block fluid flow through the catheter 12. In some embodiments, in response to the determining the presence of the occlusion based on the data sensed by the sensor 130, the communication unit 140 may wirelessly transmit the output signal to the receipt location.

To detect characteristics associated fluid pathway 126 and to evaluate whether there may be a fluid pathway occlusion or patency, or catheter extravasation or infiltration, the circuit can include an application-specific integrated circuit configured to analyze data from the sensor 130 and any additional sensor or data source.

Referring to the housing 120 of the intravenous monitoring system 100, a longitudinal axis A1 of the housing is defined by the fluid pathway 126 that extends through the housing 120. The fluid pathway 126 extends from the first end 122 to the second end 124 of the housing and along the longitudinal axis A1. A length L1 of the housing is defined between the first end 122 to the second end 124 of the housing. In some embodiments, the fluid pathway 126 and the longitudinal axis A1 are coaxial.

The inner surface of the housing forming the fluid pathway 126 is shaped as an enclosed passage through the housing 120, wherein the enclosed passage has a first opening 121 at the first end 122 of the housing and a second opening 123 at the second end 124 of the housing. In some embodiments, the inner surface of the housing forming the fluid pathway also includes one or more openings into the fluid pathway 126. The one or more openings can permit sensors to extend to the fluid pathway 126 for exposure of the sensor to a fluid in the fluid pathway while also maintaining the enclosed structure of the fluid pathway.

The housing 120 also defines a top surface 162, a bottom surface 164, and a height H1 between the top surface and the bottom surface. The housing 120 extends laterally outward in a direction away from the longitudinal axis A1 to form a first wing 166 and a second wing 168. The first wing 166 and the second wing 168 form opposing lateral ends of the housing 120, thereby defining a width W1 of the housing therebetween.

To facilitate securement of the intravenous monitoring system 100 to a patient, the housing can have a height H1 that decreases along at least a portion of the width W1 of the housing. In some embodiments, the height H1 decreases along the width W1, in a direction from the longitudinal axis A1 toward a lateral end of the housing 120. In some embodiments of the present disclosure, the height H1 decreases in a direction from the longitudinal axis A1 toward each of the lateral ends of the housing 120. The housing 120 is also shaped so that the width W1 of the housing is greater than the length L1 of the housing. Additionally, the height H1 of the housing is less than the width W1 and the length L1 of the housing.

To facilitate anchoring or securing the intravenous monitoring system 100 to a patient, a tape can be placed over at least a portion of the housing 120 and extended onto the patient such that the housing 120 is positioned between the tape and the patient. For example, a piece of medical tape can be placed over the top surface 162 of one ore more of the wings and onto the patient. In some embodiments of the present disclosure, a tape or adhesive can be applied along the bottom surface 164 of the housing so that the housing could adhere to the patient.

Tubing 14 extending from a catheter or an IV set can be coupled to the intravenous monitoring system 100 by coupling an end of a first tubing segment to the fluid pathway 126 at the first end 122 of the housing. An end of a second tubing segment can be coupled to the fluid pathway 126 at the second end 124 of the housing. Thus, a fluid moving through any of the first or second tubing segment will move through the fluid pathway 126 of the housing. In some embodiments of the present disclosure the tubing 14 can be inserted through the housing 120, e.g., through the first end 122 and the second end 124, such that the intravenous monitoring system 100 is positioned between a first end 16 of the tubing and a second end 16 of the tubing.

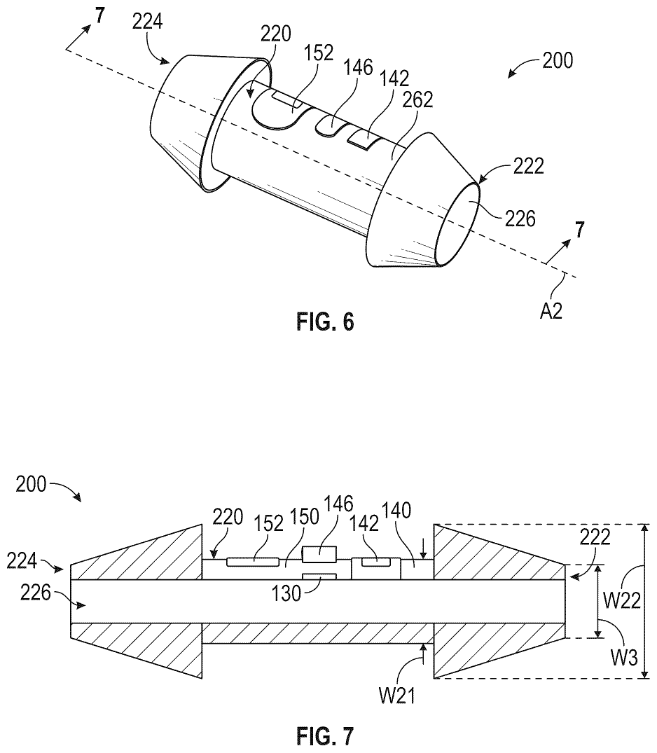

In some embodiments of the present disclosure, the intravenous monitoring system 200 is configured as a fluid coupling that can be coupled to tubing 14 extending from a catheter 12. An example of the intravenous monitoring system 200 is shown in a perspective view in FIG. 6, and a cross-section side view of the intravenous monitoring system 200 along the line 6-6 is shown in FIG. 7. The intravenous monitoring system 200 includes features that are similar to the example embodiments of the intravenous monitoring system 100, 300 described in the present disclosure. Accordingly, certain features of the intravenous monitoring system 200 are referred to with similar reference numerals used in reference to the other intravenous monitoring system 100, 300 of the present disclosure.

The intravenous monitoring system 200 includes a housing 220 having a first end 222 and a second end 224, where the second end 224 is opposite to the first end 222 of the housing. The housing 220 also includes an inner surface forming a fluid pathway 226 extending through the housing 220 and from the first end 222 to the second end 224 of the housing. The fluid pathway 226 permits a fluid, such as blood, to move through the fluid pathway 226. When the fluid moves though the fluid pathway 226, a sensor of the intravenous monitoring system 200 can sense and identify data associated with the fluid.

To sense and identify data associated with the fluid moving through the fluid pathway 226, the intravenous monitoring system 200 includes a sensor 130 and a communication unit 140. The sensor 130 is coupled to the fluid pathway 226 and can be in indirect or direct contact with the fluid in the fluid pathway 226. The sensor 130 can detect conditions and characteristics of the fluid in the fluid pathway 226, such as a change in pressure caused by the fluid. When the fluid pathway 226 is in fluid communication with a catheter, the sensor 130 can detect intravascular pressure changes such as peripheral venous pressure (PVP) waveform.

The intravenous monitoring system 200 also includes a communication unit 140 that generates an output signal based on the data sensed by the sensor 130. The output signal can include one or more various formats, including, but not limited to, a visual signal, an audible signal, a tactile signal, and/or a wirelessly transmitted output signal. In the embodiment of the intravenous monitoring system 200, the visual signal is a light source 142 that can be visible to a person near the intravenous monitoring system. Additionally, the communication unit can wirelessly transmit an output signal from the intravenous monitoring system 200 to a receipt location 20.

The intravenous monitoring system 200 includes a reset button 146 that can be activated by any of patient, a caregiver, another person, or from another system associated with the intravenous monitoring system. The reset button 146 can be used to change a characteristic of the output signal, such as, for example, silencing, turning off, snoozing, or resetting the output signal. In some embodiments, the reset button 146 can also include other functions, such as turning the intravenous monitoring system 200 off or on, or resetting/rebooting the intravenous monitoring system 200.

Within the housing 220, the intravenous monitoring system 200 contains a circuit 150 and a power supply 152. The sensor 130, the communication unit 140, the light source 142, and the power supply 152 are coupled to the circuit 150. The power supply 152 is electrically coupled to the sensor 130 and provides power to the one or more sensors. The power supply 152 may include a battery, which may be rechargeable and/or replaceable. In some embodiments, the power supply 152 may be remotely disposed from the circuit 150.

The housing 220 of the intravenous monitoring system has a longitudinal length L2 that extends between the first end 222 and the second end 224 of the housing. The fluid pathway 226 extends along the longitudinal length L2 of and through the housing 220 to define a longitudinal axis A2 of the housing. When viewed in a cross-section plane that is transverse to the longitudinal axis A2, the housing forms a cylindrical or tubular shape with an inner surface defining the fluid pathway 226.

The inner surface of the housing forming the fluid pathway 226 is shaped as an enclosed passage through the housing 220, wherein the enclosed passage has a first opening at the first end 222 of the housing and a second opening at the second end 224 of the housing. In some embodiments, the inner surface of the housing forming the fluid pathway also includes one or more openings into the fluid pathway 226. The one or more openings permit the sensor 130 to extend to the fluid pathway 226 and maintain enclosure of the fluid pathway.

The housing 120 also has an outer surface 262 with one or more openings to permit user access to features such as the output signal, e.g., the light source 142, the reset button 146, and the power supply 152. The outer surface 262 of the housing defines a width that is transverse to the longitudinal axis A2 and can vary along the length L2 of the housing. In some embodiments, the outer surface 262 of the housing is shaped as a barb having a width that decreases in a direction toward any of the first end 222 and the second end 224 of the housing.

Referring to the cross-sectional view of the intravenous monitoring system 200 in FIG. 7, for example, the housing has a first width W21 along a portion of the longitudinal length L2 between the first end 222 and the second end 224 of the housing. Proximal to the first end 222 and the second end 224 of the housing, the housing has a second width W22 that is greater than or equal to the first width W21. Further, the width of the outer surface 262 of the housing decreases along another portion of the longitudinal length L2 extending to the first end 222 and the second end 224 of the housing to a width W3 at the first end 222 and the second end 224 of the housing. Thus, the outer surface 262 of the housing can form a barb shape from the second width W22 to at any of the first end 222 and the second end 224 of the housing.

The barb shaped housing permits the intravenous monitoring system 200 to be coupled to tubing 14 of a catheter or IV set. The intravenous monitoring system 200 can be coupled to tubing 14 by inserting any of the first end 222 or the second end 224 of the housing into the lumen at the first or second end 16, 18 of the tubing (FIG. 2). The housing 220 can be inserted into the tubing 14 until the portion of the outer surface 262 with the barb shape is within the lumen of the tubing 14. In some embodiments of the present disclosure, the other of the first end 222 or the second end 224 of the housing can be inserted into the lumen at the end of another tubing so that a fluid (e.g., blood) can move through the tubing 14, through the fluid pathway 226, and then to another tubing. In some embodiments of the present disclosure, the other of the first end 222 or the second end 224 of the housing can have a shape of a connector configured to couple to another device. For example, the first end 222 or the second end 224 of the housing can have a shape of any of a Luer lock adapter, a threaded adapter, a tubular slip joint, or a needleless connector.

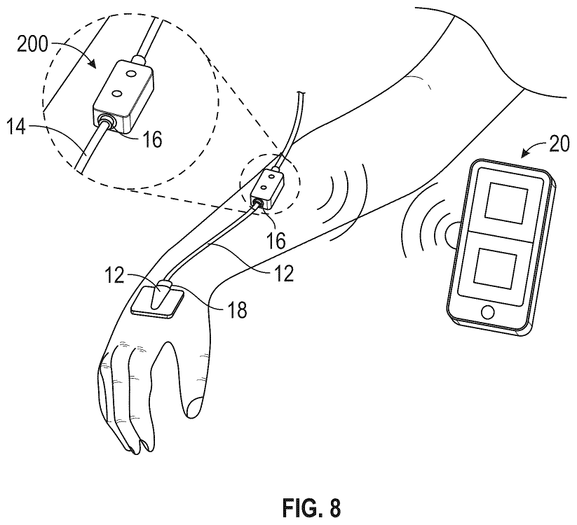

An example of the intravenous monitoring system 200 is shown in FIG. 8 with a PIVC catheter coupled to a patient. The intravenous monitoring system 200 is coupled to tubing 14 of a catheter or IV set. To fluidically couple the intravenous monitoring system 200 to the catheter 12, a first end 16 of the tubing is coupled to a first portion of the intravenous monitoring system 200 and is in fluid communication with the fluid pathway of the intravenous monitoring system, and a second end 18 of the tubing is coupled to the catheter 12. The intravenous monitoring system 200 is in communication with a clinician monitoring device as the receipt location 20. As described in the present disclosure, the communication unit 140 of the intravenous monitoring system can transmit data to and receive data from the receipt location 20.

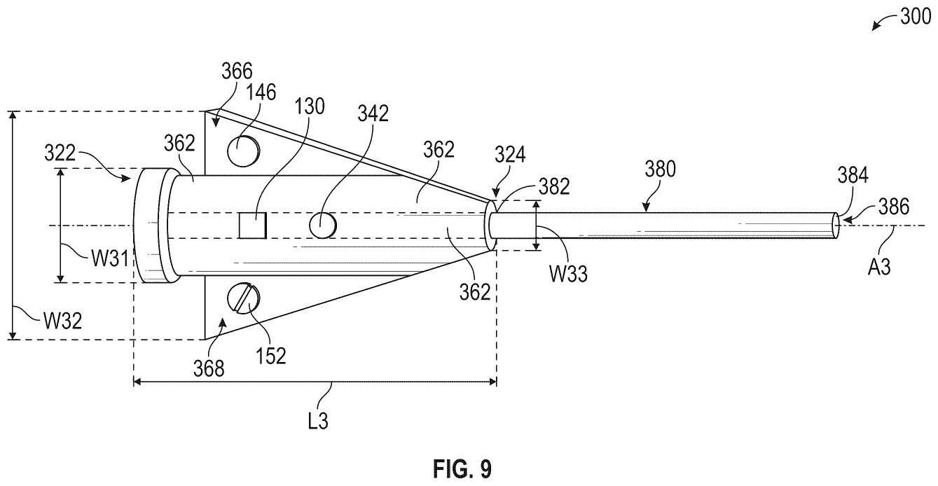

In some embodiments of the present disclosure, the intravenous monitoring system 300 is configured as a catheter comprising catheter hub for a catheter needle. An example of the intravenous monitoring system 300 is shown in a perspective view in FIG. 9. The intravenous monitoring system 300 includes features that are similar to the example embodiments of the intravenous monitoring systems 100, 200 described in the present disclosure. Accordingly, certain features of the intravenous monitoring system 300 are referred to with similar reference numerals used in reference to the other intravenous monitoring systems 100, 200.

The intravenous monitoring system 300 includes a housing 320 having a first end 322 and a second end 324, where the second end 324 is opposite to the first end 322 of the housing. The housing 320 also includes an inner surface forming a fluid pathway 326, represented by dashed lines in FIG. 9, extending through the housing 320 and from the first end 322 to the second end 324 of the housing. The fluid pathway 326 permits a fluid, such as blood, to move through the fluid pathway 326. When the fluid moves though the fluid pathway 326, a sensor of the intravenous monitoring system 300 can sense and identify data associated with the fluid.

The housing 320 also defines a top surface 361, a bottom surface, and a height between the top surface and the bottom surface. A longitudinal length L3 of the housing 320 is defined between the first end 322 and the second end 324 of the housing. The fluid pathway 326 extends along the longitudinal length L3 of and through the housing 320 to also define a longitudinal axis A3 of the housing.

The housing 320 extends laterally outward in a direction away from the longitudinal axis A3 to form a first wing 366 and a second wing 368. The first wing 366 and the second wing 368 form opposing lateral sides of the housing 320, thereby defining a width of at least a portion of the housing therebetween. In some embodiments of the present disclosure the outer surface along bottom surface of the housing 320, including the first and second wings 366, 368, can form a surface configured to engage against a patient. The outer surface along bottom surface of the housing 320 can form any of a planar surface, a concave surface, a convex surface.

The width of the housing is transverse to the longitudinal axis A3 and the width of the housing can vary along the length L3 of the housing. In some embodiments, the outer surface 362 of the housing has a first width W31 at the first end 322 of the housing, and a second width W32 at a proximal end of the first and second wings 366, 368, i.e., proximal to the first end 322 of the housing. The width of the housing decreases along the length of the first and second wings 366, 368 in a direction toward the second end 324 of the housing. Thus, in some embodiments of the present disclosure, the second width W32 is greater than the first width W31. Further, the present disclosure contemplates embodiments in which the second end 324 of the housing comprises a width that is equal to or less than the first width W31, or in some embodiments, the second end 324 of the housing comprises a width W33 that is less than the first width W31 and the second width W32.

In some embodiments of the present disclosure, a catheter needle 380 is coupled to the second end 324 of the intravenous monitoring system 300. The catheter needle 380 has a proximal end 382 coupled to the housing 320, a distal end 384 opposite to the proximal end 382, and an inner surface forming a lumen 386. The lumen 386 extends through the catheter needle 380 and is fluidically coupled to the fluid pathway 326 of the housing 320 (i.e., the catheter hub). Thus, the sensor 130 can detect conditions and characteristics of a fluid moving from the lumen 386 of the catheter needle to the fluid pathway 326 of the housing.

Although the catheter needle 380 is coupled to the housing 320, the present disclosure contemplates embodiments in which the catheter needle 380 is removably coupled to the housing 320, and in which removal of the catheter needle 380 from the housing 320 is resisted. In some embodiments, the housing 320 and the catheter needle 380 may be coupled together via any suitable coupling mechanism, including, for example, threading, a snap fit, an interference fit, friction, or an adhesive.

To sense and identify data associated with the fluid within or moving through the fluid pathway 326, the intravenous monitoring system 300 includes a sensor 130 and a communication unit. The sensor 130 is coupled to the fluid pathway 326 and can be in indirect or direct contact with the fluid in the fluid pathway 326. The sensor 130 can detect conditions and characteristics of the fluid in the fluid pathway 326, such as a change in pressure caused by the fluid. When the catheter needle 380 is intravenously positioned in a patient, the sensor 130 can detect intravascular pressure changes such as peripheral venous pressure (PVP) waveform.

The intravenous monitoring system 300 also includes a communication unit that generates an output signal based on the data sensed by the sensor 130. The output signal can include one or more various formats, including, but not limited to, a visual signal, an audible signal, a tactile signal, and/or a wirelessly transmitted output signal. In the embodiment of the intravenous monitoring system 300, the output signal is a piezo alarm 342 that can be heard by a person near the intravenous monitoring system. Additionally, the communication unit can wirelessly transmit an output signal from the intravenous monitoring system 200 to a receipt location 20.

The intravenous monitoring system 300 includes a reset button 146 that can be activated by any of patient, a caregiver, another person, or from another system associated with the intravenous monitoring system. The reset button 146 can be used to change a characteristic of the output signal, such as, for example, silencing, turning off, snoozing, or resetting the output signal. In some embodiments, the reset button 146 can also include other functions, such as turning the intravenous monitoring system 300 off or on, or resetting/rebooting the intravenous monitoring system 300.

Within the housing 320, the intravenous monitoring system 300 contains a circuit and a power supply 152. The sensor 130, the communication unit, the piezo alarm 342, and the power supply 152 are coupled to the circuit 150. The power supply 152 is electrically coupled to the sensor 130 and provides power to the one or more sensors. The power supply 152 may include a battery, which may be rechargeable and/or replaceable. In some embodiments, the power supply 152 may be remotely disposed from the circuit 150.

The inner surface of the housing forming the fluid pathway 326 is shaped as an enclosed passage through the housing 320, wherein the enclosed passage has a first opening at the first end 322 of the housing and a second opening at the second end 324 of the housing. In some embodiments, the inner surface of the housing forming the fluid pathway 326 also includes one or more openings into the fluid pathway 326. The one or more openings permit the sensor 130 to extend to the fluid pathway 326 and maintain enclosure of the fluid pathway.

The housing 320 also has an outer surface 362 with one or more openings to permit user access to features such as the output signal, e.g., the alarm 342, the reset button 146, and the power supply 352. The features of the intravenous monitoring system 300, which can include but is not limited to the alarm 342, the reset button 146, and the power supply 352, can be positioned on any portion of the housing, including the first wing 366 and the second wing 368. In the example of the intravenous monitoring system 300 in FIG. 9, the reset button 146 and the power supply 352 are positioned on the first wing 366 and the second wing 368, respectively. The sensor 130 and the piezo alarm 342 are positioned in a portion of the housing that is between the first wing 366 and the second wing 368.

Referring to FIG. 1, an example receipt location 20 is illustrated, according to some embodiments. In some embodiments, the receipt location 20 may include a clinician monitoring device. Examples of the clinician monitoring device may include a computing device, a mobile phone, a smartphone, a tablet computer, a laptop computer, a desktop computer, a medical device, or a connected device (e.g., a smartwatch, smart glasses, or any other connected device). In some embodiments, in addition to or as an alternative to the monitoring device providing the alert, the clinician monitoring device may provide the alert. In some embodiments, the clinician monitoring device may include a pump, which may be coupled to the intravenous monitoring system or coupled to tubing 14 coupled with the intravenous monitoring system. The pump can be configured to infuse the intravenous monitoring system in response to receipt of the output signal.

In some embodiments, the clinician monitoring device may include a display screen 22, which may provide the alert. In some embodiments, the alert may include a phrase such as, for example, “Partial Occlusion” or “Full Occlusion.” In some embodiments, the alert may include a visual cue on the display screen 22, such as a portion 24 of the display screen that lights up or changes color. In some embodiments, the portion 24 of the display screen may blink or change a rate of blinking to provide the alert. In some embodiments, an electronic health record that may be presented on the display screen of the clinician monitoring device.

In some embodiments, one or more intravenous monitoring system 100, 200, 300 may be connected to one or more receipt location 20, one or more, clinician monitoring devices, and/or one or more computing system via a network. In these and other embodiments, the network may include a wired or wireless network, and may have any suitable configuration, such as a star configuration, a token ring configuration, or other configurations. Furthermore, in some embodiments, the network may include an Ethernet network, a local area network (LAN), a wide area network (WAN) (e.g., the Internet), and/or other interconnected data paths across which multiple devices may communicate. In some embodiments, the network may include a peer-to-peer network. In some embodiments, the network may also be coupled to or include portions of a telecommunications network that may enable communication of data in a variety of different communication protocols.

In some embodiments, the network may include BLUETOOTH® communication networks and/or cellular communications networks for sending and receiving data including via short messaging service (SMS), multimedia messaging service (MMS), hypertext transfer protocol (HTTP), direct data connection, wireless application protocol (WAP), e-mail, etc. The network may enable communication via a standard-based protocol such as smart energy profile (SEP), Echonet Lite, OpenADR, or another suitable protocol, e.g., wireless fidelity (Wi-Fi), ZigBee, Z-Wave, HomePlug Green, etc.

In some embodiments, the communication unit 140 may be configured to transmit data to and receive data from the receipt location 20 or a clinician monitoring devices via the network. In some embodiments, the communication unit 140 may also be configured to transmit and receive data from a display screen and/or an electronic health record. In some embodiments, the display screen may include or correspond to the display screen 22 described with respect to the receipt location 20.

In some embodiments, the communication unit 140 may include a cellular communications transceiver for sending and receiving data over a cellular communications network including via SMS, MMS, HTTP, direct data connection, WAP, e-mail, or another suitable type of electronic communication. The communication unit 140 may also provide other conventional connections to the network for distribution of files or media objects using standard network protocols including transmission control protocol/internet protocol (TCP/IP), HTTP, HTTP secure (HTTPS), and simple mail transfer protocol (SMTP).

Various examples of aspects of the subject technology are described below for convenience. These are provided as examples and do not limit the subject technology. Further, the examples may be combined in any combination, and placed into respective embodiments.

An intravenous monitoring system comprising a housing comprising a first end, a second end opposite to the first end, a longitudinal axis formed between the first end and the second end, and an inner surface forming a fluid pathway extending through the housing and configured for coupling with a catheter, a sensor coupled to the fluid pathway to detect data for a fluid in the fluid pathway, a communication unit coupled to the sensor for conveying data sensed by the sensor, and a power source electrically coupled to the sensor.

Wherein the fluid pathway extends through the first end of the housing and the second end of the housing.

Wherein the first end of the housing is configured to couple with an end of a first tubing, and the second end of the housing is configured to couple with an end of a second tubing.

Wherein an outer surface of the housing is shaped as a barb having a width that decreases in a direction toward any of the first end and the second end of the housing.

The intravenous monitoring system further comprising a catheter needle coupled to the fluid pathway and extending in a direction away from the housing.

Wherein a portion of the fluid pathway extending through the first end of the housing is configured to fluidically couple with an end of a tubing, and the fluid pathway extending through the second end of the housing is configured to fluidically couple with the catheter needle.

Wherein tubing extends through the fluid pathway of the housing such that the housing is positioned between a first end of the tubing and a second end of the tubing.

The intravenous monitoring system further comprising a wing coupled to the housing and extending laterally outward in a direction away from the housing.

Wherein the wing comprises a width in a direction that is transverse to the longitudinal axis and defined between a proximal end of the wing at the housing and a distal end of the wing, and a length in a direction along the longitudinal axis and defined between a first end of the wing and a second end of the wing.

Wherein the width of the wing decreases in a direction from the first end of the wing toward the second end of the wing, and wherein the second end of the wing is oriented toward the second end of the housing.

Wherein the sensor is a pressure sensor configured to detect peripheral venous pressure waveform of a fluid in the fluid pathway.

The intravenous monitoring system further comprising a circuit coupled to the sensor and configured to analyze the data from the sensor.

Wherein the communication unit comprises a wireless transmitter coupled to the sensor and configured to transmit data from the sensor.

Wherein the communication unit comprises any of a visual or audible alarm.

The intravenous monitoring system further comprising further comprising a reset button.

An intravenous monitoring system comprising a catheter comprising catheter hub and a catheter needle, the catheter hub comprising a housing having an inner surface forming a fluid pathway extending through the housing, and the catheter needle having a proximal end coupled to the housing, a distal end, and an inner surface forming a lumen extending through the catheter needle and fluidically coupled to the fluid pathway, a sensor coupled to the housing to detect data for a fluid in any of the fluid pathway and the lumen, a communication unit coupled to the housing for conveying data sensed by the sensor, and a power source coupled to the housing and electrically coupled to the sensor.

The intravenous monitoring system further comprising a wing coupled to the housing and extending laterally outward in a direction away from the housing.

Wherein the wing comprises a width in a direction that is transverse to the longitudinal axis and defined between a proximal end of the wing at the housing and a distal end of the wing, and a length in a direction along the longitudinal axis and defined between a first end of the wing and a second end of the wing.

Wherein the width of the wing decreases in a direction from the first end of the wing toward the second end of the wing, and wherein the second end of the wing is oriented toward the second end of the housing.

Wherein the sensor is a pressure sensor configured to detect peripheral venous pressure waveform of a fluid in the fluid pathway.

The intravenous monitoring system further comprising a circuit coupled to the sensor and configured to analyze the data from the sensor.

Wherein the communication unit comprises a wireless transmitter coupled to the sensor and configured to transmit data from the sensor.

Wherein the communication unit comprises any of a visual or audible alarm.

The intravenous monitoring system further comprising further comprising a reset button.

An intravenous monitoring system comprising a catheter comprising catheter hub and a catheter needle, the catheter hub comprising a housing having an inner surface forming a fluid pathway extending through the housing, and the catheter needle having a proximal end coupled to the housing, a distal end, and an inner surface forming a lumen extending through the catheter needle and fluidically coupled to the fluid pathway, a sensor coupled to the housing to detect data for a fluid in any of the fluid pathway and the lumen, a communication unit coupled to the housing for conveying data sensed by the sensor, a power source coupled to the housing and electrically coupled to the sensor.

The intravenous monitoring system further comprising a wing coupled to the housing and extending laterally outward in a direction away from the housing.

Wherein the wing comprises a proximal end coupled to the housing and a distal end opposite to the proximal end, and a width defined from the proximal end to the distal end of the wing, and wherein the width of the wing decreases in a direction toward the catheter.

A method for providing intravenous monitoring system comprising providing a housing comprising a first end, a second end opposite to the first end, and an inner surface forming a fluid pathway extending through the housing and configured for fluidically coupling with a catheter, providing a sensor coupled to the fluid pathway to detect data for a fluid in the fluid pathway, providing a communication unit coupled to the sensor for conveying data sensed by the sensor, and providing a power source electrically coupled to the sensor.

The method for providing intravenous monitoring system further comprising providing the housing between a catheter needle and a tubing.

The method for providing intravenous monitoring system further comprising providing a circuit coupled to the sensor and configured for analyzing the data from the sensor.

Wherein analyzing the data from the sensor comprises comparing the data to a pre-determined alarm limit.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An intravenous monitoring system comprising:
a housing comprising a first end, a second end opposite to the first end, a longitudinal axis formed between the first end and the second end, and an inner surface forming a fluid pathway extending through the housing and configured for coupling with a catheter;
a sensor coupled to the fluid pathway to detect data for a fluid in the fluid pathway;
a communication unit coupled to the sensor for conveying data sensed by the sensor; and
a power source electrically coupled to the sensor.

2. The intravenous monitoring system of claim 1, wherein the fluid pathway extends through the first end of the housing and the second end of the housing.

3. The intravenous monitoring system of claim 2, wherein the first end of the housing is configured to couple with an end of a first tubing, and the second end of the housing is configured to couple with an end of a second tubing.

4. The intravenous monitoring system of claim 1, wherein an outer surface of the housing is shaped as a barb having a width that decreases in a direction toward any of the first end and the second end of the housing.

5. The intravenous monitoring system of claim 1, further comprising a catheter needle coupled to the fluid pathway and extending in a direction away from the housing.

6. The intravenous monitoring system of claim 5, wherein a portion of the fluid pathway extending through the first end of the housing is configured to fluidically couple with an end of a tubing, and the fluid pathway extending through the second end of the housing is configured to fluidically couple with the catheter needle.

7. The intravenous monitoring system of claim 1, wherein tubing extends through the fluid pathway of the housing such that the housing is positioned between a first end of the tubing and a second end of the tubing.

8. The intravenous monitoring system of claim 1, further comprising a wing coupled to the housing and extending laterally outward in a direction away from the housing.

9. The intravenous monitoring system of claim 8, wherein the wing comprises a width in a direction that is transverse to the longitudinal axis and defined between a proximal end of the wing at the housing and a distal end of the wing, and a length in a direction along the longitudinal axis and defined between a first end of the wing and a second end of the wing.

10. The intravenous monitoring system of claim 9, wherein the width of the wing decreases in a direction from the first end of the wing toward the second end of the wing, and wherein the second end of the wing is oriented toward the second end of the housing.

11. The intravenous monitoring system of claim 1, wherein the sensor is a pressure sensor configured to detect peripheral venous pressure waveform of a fluid in the fluid pathway.

12. The intravenous monitoring system of claim 1, further comprising a circuit coupled to the sensor and configured to analyze the data from the sensor.

13. The intravenous monitoring system of claim 1, wherein the communication unit comprises a wireless transmitter coupled to the sensor and configured to transmit data from the sensor.

14. The intravenous monitoring system of claim 1, wherein the communication unit comprises any of a visual or audible alarm.

15. The intravenous monitoring system of claim 1, further comprising further comprising a reset button.

16. An intravenous monitoring system comprising:
a catheter comprising catheter hub and a catheter needle, the catheter hub comprising a housing having an inner surface forming a fluid pathway extending through the housing, and the catheter needle having a proximal end coupled to the housing, a distal end, and an inner surface forming a lumen extending through the catheter needle and fluidically coupled to the fluid pathway;
a sensor coupled to the housing to detect data for a fluid in any of the fluid pathway and the lumen;
a communication unit coupled to the housing for conveying data sensed by the sensor; and
a power source coupled to the housing and electrically coupled to the sensor.

17. The intravenous monitoring system of claim 16, further comprising a wing coupled to the housing and extending laterally outward in a direction away from the housing.

18. The intravenous monitoring system of claim 17, wherein the wing comprises a proximal end coupled to the housing and a distal end opposite to the proximal end, and a width defined from the proximal end to the distal end of the wing, and wherein the width of the wing decreases in a direction toward the catheter.

19. A method for providing intravenous monitoring system comprising:
providing a housing comprising a first end, a second end opposite to the first end, and an inner surface forming a fluid pathway extending through the housing and configured for fluidically coupling with a catheter;
providing a sensor coupled to the fluid pathway to detect data for a fluid in the fluid pathway;
providing a communication unit coupled to the sensor for conveying data sensed by the sensor; and
providing a power source electrically coupled to the sensor.

20. The method of claim 19, further comprising providing the housing between a catheter needle and a tubing.

21. The method of claim 19, further comprising providing a circuit coupled to the sensor and configured for analyzing the data from the sensor.

22. The method of claim 21, wherein analyzing the data from the sensor comprises comparing the data to a pre-determined alarm limit.
